(19) United States
(12) Patent Application Publication
(10) Pub. No.: US 2026/0232958 A1
(43) Pub. Date: Aug. 13, 2026

(54) PROCEDURE TOOL MODULE FOR VASCULAR INTERVENTIONAL PROCEDURE DEVICE, PROCEDURE TOOL MODULE SET HAVING THE SAME, AND VASCULAR INTERVENTIONAL PROCEDURE DEVICE SET HAVING THE SAME

(71) Applicant: PERAZAH INC., Ansan-si, Gyeonggi-do (KR)

(72) Inventors: Hwan Taek RYU, Gyeonggi-do (KR); Jong Tae SEO, Gyeonggi-do (KR)

(73) Assignee: PERAZAH INC., Ansan-si, Gyeonggi-do (KR)

(21) Appl. No.: 19/154,456

(22) PCT Filed: Feb. 6, 2024
(86) PCT No.: PCT/KR2024/095082

§ 371 Date: Aug. 7, 2025

(30) Foreign Application Priority Data
Feb. 7, 2023 (KR) 10-2023-0016229

(51) Int. Cl. A61M 25/01; A61M 25/00; A61M 25/09

(52) U.S. Cl. CPC A61M 25/0113; A61M 25/09041; A61M 2025/0004; A61M 2210/12

(57) ABSTRACT

A procedure tool module includes: a base assembly; a bobbin assembly mounted to the base assembly to be rotatable about a bobbin axis and to be movable in a vertical moving direction parallel to the bobbin axis, and configured such that a flexible wire-type or tube-type procedure tool insertable into a blood vessel is wound in a circumferential direction with respect to the bobbin axis; and a vertical moving mechanism configured to raise and lower the bobbin assembly in the vertical moving direction relative to the base assembly in association with an amount of rotation of the bobbin assembly about the bobbin axis. The bobbin assembly and the base assembly define an accommodation space therebetween such that the procedure tool is disposed in the accommodation space, the length of which in the vertical moving direction changes according to the vertical movement of the bobbin assembly relative to the base assembly.

### TECHNICAL FIELD

The present disclosure relates to a procedure tool module. More specifically, the present disclosure relates to a procedure tool module for controlling a flexible wire-type or tube-type procedure tool insertable into a blood vessel.

### BACKGROUND

A vascular intervention procedure is a minimally invasive procedure performed for the treatment of vascular diseases or cancer. It is typically conducted under X-ray fluoroscopy. In this procedure, a thin catheter with a diameter of several millimeters or less is percutaneously inserted through a blood vessel to reach a lesion site and deliver treatment to a target organ. Representative vascular intervention procedures currently performed in Korea and worldwide include, for example, trans-arterial chemoembolization (TACE) for liver cancer, percutaneous transluminal angioplasty, and artificial vascular stent placement for aortic disease.

Blood vessels are mostly divided into multiple branches or have curved structures.

Therefore, in order to prevent damage to blood vessels, vascular intervention procedures use overlapping procedure tools with multiple diameter stages, called a co-axial system of catheters and guide wires. At this time, because the blood vessels have branching points where a blood vessel is divided into several branches or curved sections, an operator should manually steer the catheters and guide wires accurately according to the directions of the blood vessels for insertion.

### DISCLOSURE

### Technical Problem

A procedure tool such as a catheter or a guide wire may be accommodated in a wound state in a procedure tool module. As a portion of the procedure tool module rotates, the procedure tool is unwound or wound, so that the procedure tool is inserted into or extracted from a blood vessel. When the procedure tool is extracted from the blood vessel, a vascular-side end of the procedure tool retracts, and when the procedure tool is inserted into the blood vessel, the vascular-side end moves forward. However, when a tool accommodation space inside the procedure tool module is excessively larger than the volume occupied by the procedure tool accommodated therein, a delay may occur in the movement of the procedure tool during a transition between the extraction and insertion of the procedure tool. For example, an operator may rotate an operating portion of the procedure tool module in one direction or in the opposite direction to advance or retract the procedure tool. However, during a transition period in which the rotation direction of the operating portion is switched, the procedure tool may not move. In order to advance the procedure tool into a target blood vessel, the operator may repeatedly perform advancing or retracting operations on the end of the procedure tool. When a delay occurs between the advancing and retracting operations, it may become difficult for the operator to insert the procedure tool into the target blood vessel.

Embodiments of the present disclosure solve the above-described problems of the prior art. Specifically, embodiments of the present disclosure provide a procedure tool module configured to prevent or minimize a delay in the movement of a procedure tool during a transition between extraction and insertion of the procedure tool when the procedure tool module is used.

### Technical Solution

According to an embodiment of the present disclosure, a procedure tool module includes: a base assembly: a bobbin assembly mounted to the base assembly to be rotatable about a bobbin axis and to be movable in a vertical moving direction parallel to the bobbin axis, the bobbin assembly being configured such that a flexible wire-type or tube-type procedure tool insertable into a blood vessel is wound in a circumferential direction with respect to the bobbin axis; and a vertical moving mechanism configured to raise and lower the bobbin assembly in the vertical moving direction with respect to the base assembly in association with an amount of rotation of the bobbin assembly about the bobbin axis. The bobbin assembly and the base assembly define an accommodation space therebetween such that the procedure tool is disposed in the accommodation space, the bobbin assembly includes a first terminal-end-surface defining a terminal end of the accommodation space in a first direction of the vertical moving direction, and the base assembly includes a second terminal-end-surface defining a terminal end of the accommodation space in a second direction opposite to the first direction of the vertical moving direction.

A length of the accommodation space in the vertical moving direction between the terminal end in the first direction and the terminal end in the second direction may change according to vertical movement of the bobbin assembly in the vertical moving direction with respect to the base assembly.

The length of the accommodation space in the vertical moving direction may increase when the bobbin assembly moves in the first direction with respect to the base assembly, and decrease when the bobbin assembly moves in the second direction with respect to the base assembly.

The bobbin assembly may be configured to move in the first direction with respect to the base assembly when the bobbin assembly rotates in a first rotation direction about the bobbin axis to wind the procedure tool within the bobbin assembly.

The bobbin assembly may be configured to move in a second direction with respect to the base assembly when the bobbin assembly rotates in a second rotation direction opposite to the first rotation direction to unwind the procedure tool from the bobbin assembly.

The accommodation space may extend in a circumferential direction with respect to the bobbin axis of the bobbin assembly.

The bobbin assembly may include a first wall surface defining a terminal end of the accommodation space in a radially inner direction with respect to the bobbin axis.

The accommodation space may extend in a circumferential direction with respect to the bobbin axis, and the procedure tool may be configured to be wound helically on the first wall surface.

The bobbin assembly may include a second wall surface defining a terminal end of the accommodation space in a radially outer direction with respect to the bobbin axis.

The base assembly may include a protruding wall configured to be at least partially accommodated between the first wall surface and the second wall surface and to define the second terminal-end-surface.

The first terminal-end-surface, the first wall surface, and the second wall surface may extend in a circumferential direction with respect to the bobbin axis, and the protruding wall may extend in the circumferential direction with respect to the bobbin axis.

The base assembly may include a guide passage connected to the accommodation space to guide the procedure tool.

The procedure tool module may further include a rotation body rotatably mounted to the base assembly about the bobbin axis, and the bobbin assembly may be rotatable about the bobbin axis together with the rotation body and may be mounted to the rotation body to be movable in the vertical moving direction parallel to the bobbin axis with respect to the rotation body.

The vertical moving mechanism may further include a vertical moving portion configured to move together with the bobbin assembly in the vertical moving direction, and a vertical moving guide portion configured to move the vertical moving portion in the vertical moving direction with respect to the base assembly.

One of the vertical moving guide portion and the vertical moving portion may include a vertical moving groove extending helically about the bobbin axis, and the other of the vertical moving guide portion and the vertical moving portion may include a vertical moving protrusion configured to be at least partially accommodated in the vertical moving groove and to be guided along the vertical moving groove when the vertical moving guide portion and the vertical moving portion rotate relative to each other.

The vertical moving mechanism may include a vertical moving guide portion including a rotation pin extending along the bobbin axis from the rotation body and coupled to the bobbin assembly to be movable in the vertical moving direction, and a vertical moving portion rotatably coupled to the bobbin assembly about the bobbin axis and including a pin hole through which the rotation pin passes. One of an outer circumferential surface of the rotation pin and an inner circumferential surface of the pin hole may include a vertical moving groove extending helically about the bobbin axis, and the other of the outer circumferential surface of the rotation pin and the inner circumferential surface of the pinhole may include a vertical moving protrusion configured to be at least partially accommodated in the vertical moving groove and to be guided along the vertical moving groove when the vertical moving guide portion and the vertical moving portion rotate relative to each other. As the rotation pin rotates and the vertical moving protrusion is guided along the vertical moving groove, the vertical moving portion may be configured to move in the vertical moving direction with respect to the base assembly, and the bobbin assembly may be configured to move in the vertical moving direction together with the vertical moving portion while rotating about the bobbin axis.

The bobbin assembly may include a pin accommodation portion configured to allow movement of the rotation pin in the vertical moving direction with respect to the bobbin assembly while restricting rotation of the rotation pin about the bobbin axis with respect to the bobbin assembly.

The bobbin assembly may include a medical torque device accommodation portion configured to accommodate a medical torque device that clamps one end of the procedure tool, and an opening configured to expose at least a portion of the medical torque device accommodation portion to an outside of the bobbin assembly.

The procedure tool module may further include a bobbin driver coupled to the base assembly and configured to rotate the bobbin assembly. The base assembly may include a base shaft extending along a base axis perpendicular to the bobbin axis, and the base shaft may include an access hole that communicates with the accommodation space and opens in a direction of the base axis.

A procedure tool module set includes the procedure tool module, and a flexible wire-type or tube-type procedure tool wound within the bobbin assembly of the procure tool module and insertable into a blood vessel.

A vascular intervention procedure device may include the procedure tool module, and a platform to which the procedure tool module is detachably coupled, the platform being configured to transmit a rotational torque to rotate the bobbin assembly to the procedure tool module.

The platform may be configured to rotate the base assembly about a base axis perpendicular to the bobbin axis.

### Advantageous Effects

According to embodiments of the present disclosure, a delay in the movement of a procedure tool may be prevented or minimized during a transition between extraction and insertion of the procedure tool when the procedure tool module is used.

### DESCRIPTION OF DRAWINGS

FIG. 1 schematically illustrates an example of insertion and rotation of a procedure tool in a vascular intervention procedure.

FIG. 2 is a perspective view of a procedure tool module for a vascular intervention procedure according to an embodiment.

FIG. 3 is a schematic cross-sectional view of the procedure tool module according to an embodiment.

FIG. 4 illustrates a releasing operation of the procedure tool in the procedure tool module of FIG. 3.

FIG. 5 illustrates a winding operation of the procedure tool in the procedure tool module of FIG. 3.

FIG. 6A illustrates the movement of the procedure tool in the accommodation space during an advancing process of the procedure tool in a comparative example.

FIG. 6B illustrates the movement of the procedure tool in the accommodation space during a retracting process of the procedure tool in a comparative example.

FIG. 6C illustrates a radial movement of the procedure tool within the accommodation space in a comparative example.

FIG. 7 is an exploded perspective view of the procedure tool module according to an embodiment.

FIG. 8 is an rear exploded perspective view of the procedure tool module according to an embodiment.

FIG. 9 is a cross-sectional view taken along line I-I′ of FIG. 2.

FIG. 10 is an exploded perspective view of a vertical moving mechanism according to an embodiment.

FIG. 11 illustrates a rotation and descending operation of the bobbin assembly.

FIG. 12 illustrates a rotation and ascending operation of the bobbin assembly.

FIG. 13 is an exploded perspective view of a medical torque device according to an embodiment.

FIG. 14 is a cross-sectional view taken along line II-II′ in FIG. 2.

FIG. 15 is a perspective view illustrating a platform of a vascular intervention device according to an embodiment.

FIG. 16 is a perspective view illustrating an example of a transferring portion illustrated in FIG. 11.

FIG. 17 is a bottom perspective view illustrating a power transmitting portion of the transferring portion illustrated in FIG. 16.

FIG. 18 is a cross-sectional view taken along line III-III′ in FIG. 17.

FIG. 19 illustrates a vertical moving mechanism in a first modification.

FIG. 20 illustrates a vertical moving mechanism in a second modification.

FIG. 21 illustrates a vertical moving mechanism in a third modification.

FIG. 22 illustrates a vertical moving mechanism in a fourth modification.

FIG. 23 illustrates a vertical moving mechanism in a fifth modification.

### MODE FOR INVENTION

Embodiments of the present disclosure are exemplified for the purpose of explaining the technical idea of the present disclosure. The scope of rights according to the present disclosure is not limited to the embodiments presented below or the specific descriptions of these embodiments.

All technical and scientific terms used in the present disclosure have meanings generally understood by those of ordinary skill in the art to which the present disclosure pertains, unless otherwise defined. All terms used in the present disclosure are selected for describing the present disclosure more clearly, and are not selected to limit the scope of the rights according to the present disclosure.

As used in the present disclosure, expressions such as “comprising”, “including”, “having”, and the like are to be understood as open-ended terms that imply the possibility of encompassing other embodiments, unless otherwise mentioned in the phrase or sentence containing such expressions.

As used in the present disclosure, singular expressions may encompass plural meanings unless otherwise stated, and this also applies to the singular expressions recited in the claims.

As used in the present disclosure, expressions such as “first” and “second” used in the present disclosure are used to distinguish a plurality of elements from each other, and are not intended to limit the order or importance of the corresponding elements.

In the present disclosure, where it is mentioned in the present disclosure that one element is “connected” or “joined” to another element, it is to be understood that said one element may be directly connected to said another element, or may be connected to said another element via a new additional element.

The dimensional and numerical values described in the present disclosure are not limited only to the dimensional and numerical values that are described herein. Unless specified otherwise, the dimensional and numerical values may be understood to mean the described values and equivalent ranges that include the values. For example, a dimension “xx mm” described herein may be understood to include “about xx mm.”

As used in the present disclosure, the direction indicators, such as “upward” and “upper,” refer to the direction in which the bobbin assembly is positioned relative to the base assembly in the attached drawings, and the direction indicators, such as “downward” and “down,” refer to the opposite direction. The bobbin assembly and the base assembly illustrated in the attached drawings may be oriented differently, and the direction indicators may be interpreted accordingly.

Hereinafter, embodiments of the present disclosure will be described with reference to the accompanying drawings. In the accompanying drawings, the same or corresponding components are assigned the same reference numerals. In addition, in the description of the following embodiments, duplicate descriptions of the same or corresponding components may be omitted. However, even if descriptions of components are omitted, it is not intended that such components are not included in any of the embodiments.

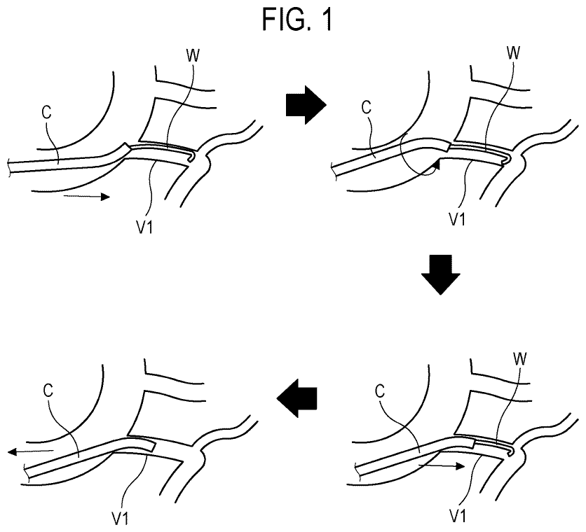

FIG. 1 schematically illustrates an example of insertion and rotation of a procedure tool in a vascular intervention procedure.

The embodiment of the present disclosure and the embodiment illustrated in the drawings relate to a procedure tool module used to transfer and rotate procedure tools used in vascular intervention procedures and to introduce the procedure tools into a target blood vessel. The procedure tool module according to the embodiments is used in a vascular intervention procedure employing a catheter, a guide wire, a microcatheter, and a micro guide wire. In the present disclosure, the catheter, the guide wire, the microcatheter, and the micro guide wire are referred to as procedure tools.

The catheter is a flexible tube that is inserted into a target blood vessel. The guide wire is inserted into the catheter so as to guide the catheter to the target blood vessel. The microcatheter is a flexible tube that can be inserted into the catheter. The microcatheter enters a narrower target blood vessel that the catheter cannot enter, and is used to inject a drug into the narrower target blood vessel or to aspirate blood clots. The micro guide wire has a smaller diameter than the guide wire and is used to guide the microcatheter into the narrower target blood vessel. The micro guide wire is inserted into the microcatheter. FIG. 1 schematically illustrates an example of insertion and rotation of a procedure tool in a procedure tool module.

Referring to FIG. 1, an example in which a catheter is delivered to a target blood vessel using a catheter and a guide wire by a procedure tool module according to an embodiment is described. The procedure tool module may transfer a catheter c and a guide wire w in order to advance a catheter c and a guide wire w to a region near a first target blood vessel v1. The procedure tool module may transfer and rotate the guide wire w in order to introduce the guide wire w into the first target blood vessel v1. In addition, the procedure tool module may rotate the catheter c along with the transfer and rotation of the guide wire w. Once the guide wire w is introduced into the first target blood vessel v1, the procedure tool module may introduce the catheter c into the first target blood vessel v1 along the guide wire w. When the catheter c reaches the first target blood vessel v1, the guide wire w is removed from the catheter c. The catheter c may be used to inject a drug into the first target blood vessel v1 or to aspirate a thrombus in the first target blood vessel v1. Hereinafter, a procedure tool module that implements the movement (transfer and rotation) of the procedure tool will be described.

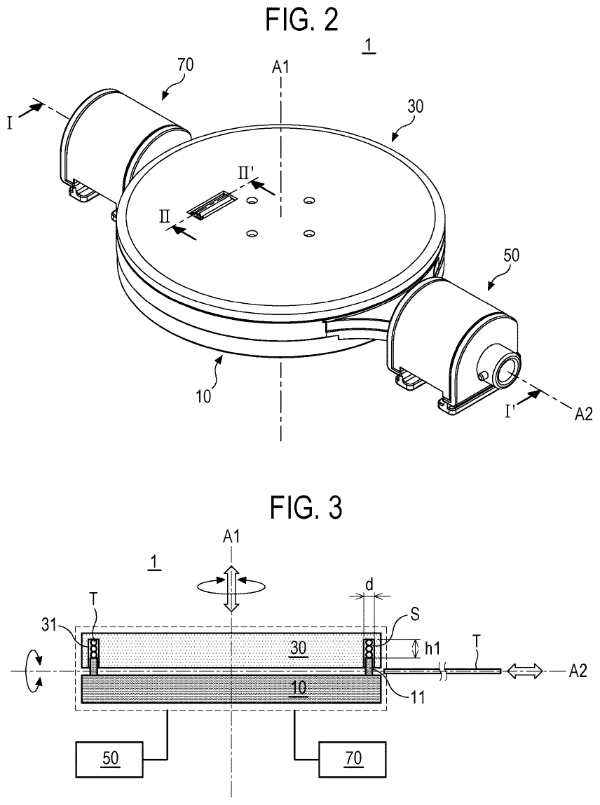

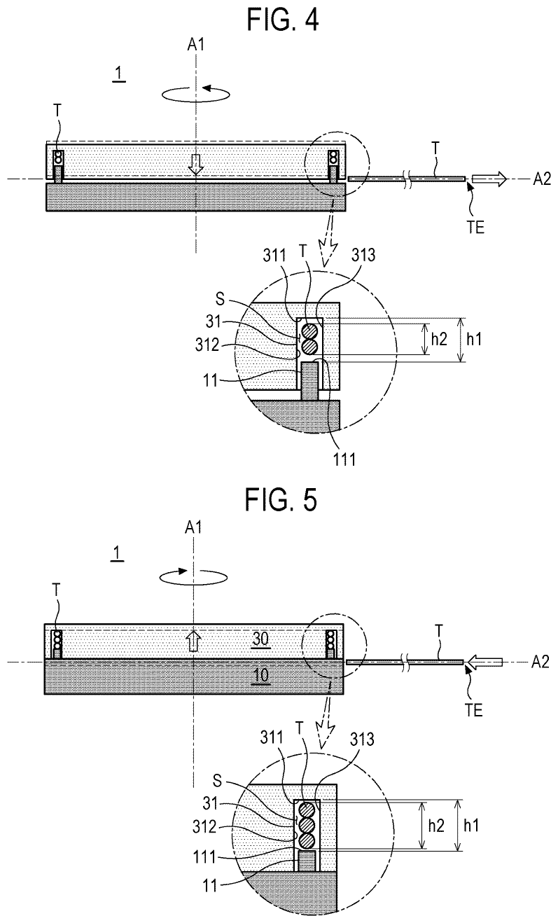

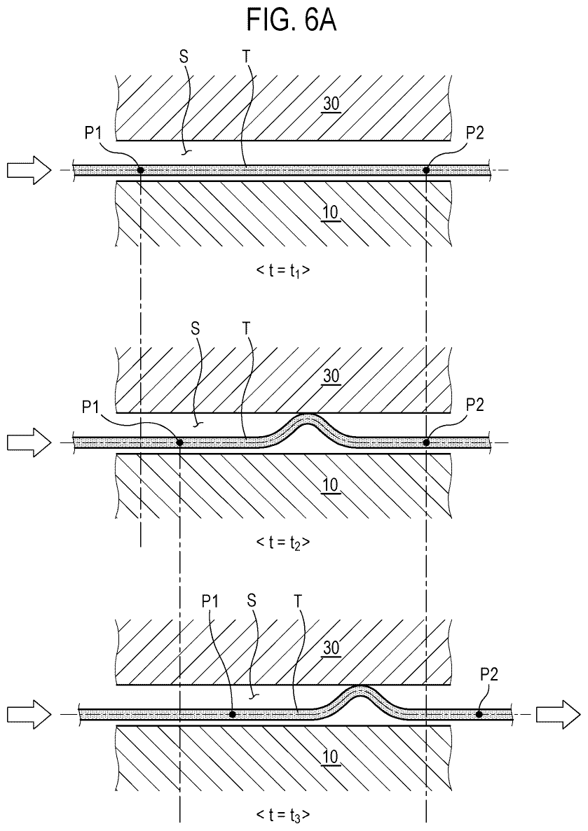

FIG. 2 is a perspective view of a procedure tool module 1 for a vascular intervention procedure according to an embodiment. FIG. 3 is a schematic cross-sectional view of the procedure tool module 1 according to an embodiment. FIG. 4 illustrates a releasing operation of the procedure tool T in the procedure tool module 1 of FIG. 3. FIG. 5 illustrates a winding operation of the procedure tool T in the procedure tool module 1 of FIG. 3.

Referring to FIGS. 2 and 3, the procedure tool module 1 includes a base assembly 10 and a bobbin assembly 30. The bobbin assembly 30 is coupled to the base assembly 10 to be rotatable about a bobbin axis Al. The procedure tool module 1 may include a base driver 50 configured to rotate the base assembly 10. The base driver 50 may rotate the base assembly 10 about a base axis A2, which is perpendicular to the bobbin axis A1. As the base assembly 10 rotates, the procedure tool T rotates. The procedure tool module 1 may include a bobbin driver 70 configured to rotate the bobbin assembly 30 relative to the base assembly 10 about the bobbin axis A1. As the bobbin assembly 30 rotates, the procedure tool T is transferred.

Referring to FIG. 3, the bobbin assembly 30 is configured such that a flexible wire-type or tube-type procedure tool T, which is insertable into a blood vessel, is wound in the circumferential direction with respect to the bobbin axis A1. The procedure tool T is disposed in an accommodation space S defined between the base assembly 10 and the bobbin assembly 30. The accommodation space S extends in the circumferential direction with respect to the bobbin axis A1, and a portion of the procedure tool T is wound in the accommodation space S. The bobbin assembly 30 may have an accommodation groove 31 that defines at least a portion of the accommodation space S.

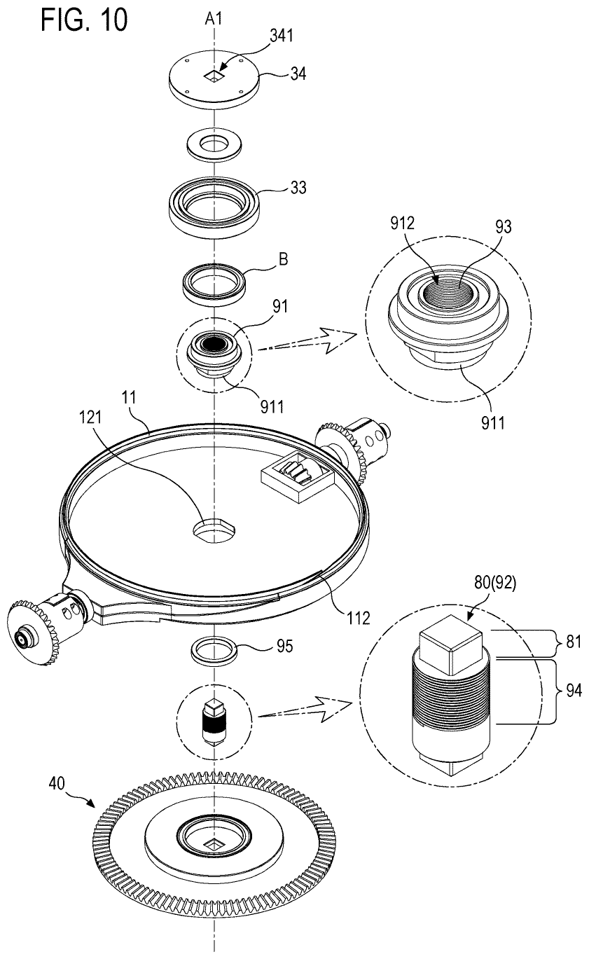

The procedure tool T is extracted from or inserted into the procedure tool module 1 according to the rotation of the bobbin assembly 30 about the bobbin axis Al. That is, the procedure tool T is inserted into or extracted from a blood vessel according to the rotation of the bobbin assembly 30 about the bobbin axis A1. Although not illustrated, the procedure tool T disposed in the accommodation space S may exit the procedure tool module 1 through a guide passage (e.g., the guide passage 112 of FIG. 10) defined in the base assembly 10.

Referring to FIG. 4, as the bobbin assembly 30 rotates in one direction (e.g., clockwise) relative to the base assembly 10, a portion of the procedure tool T wound within the bobbin assembly 30 (hereinafter, referred to as the “wound portion”) is partially unwound and extracted to the outside of the procedure tool module 1. Accordingly, a terminal end TE of the procedure tool T advances and is inserted into a target blood vessel. Referring to FIG. 5, as the bobbin assembly 30 rotates in a direction opposite to FIG. 4 (e.g., counterclockwise) relative to the base assembly 10, a portion of the procedure tool T is wound into the accommodation space S. Accordingly, a portion of the procedure tool T that has been located outside the procedure tool module 1 is inserted into the procedure tool module 1. As a result, the terminal end TE of the procedure tool T retreats and is extracted from the blood vessel.

The procedure tool T is helically wound and accommodated in the accommodation space S. The height of the wound portion along the bobbin axis Al (i.e., the vertical direction in the drawings) is proportional to the number of windings of the procedure tool T. For example, referring to FIG. 3, when the procedure tool T is wound three times within the bobbin assembly 30, the height h2 of the wound portion corresponds to three times the thickness of the cross-section of the procedure tool T. Referring to FIG. 4, when the procedure tool T is wound two times within the bobbin assembly 30, the height h2 of the wound portion corresponds to two times the thickness of the cross-section of the procedure tool T.

The bobbin assembly 30 may include a first terminal-end-surface 311. The first terminal-end-surface 311 may define the upper end of the accommodation space S. Referring to FIGS. 4 and 5, the first terminal-end-surface 311 may be provided by a bottom surface of the accommodation groove 31 provided in the bobbin assembly 30. The base assembly 10 may include a second terminal-end-surface 111. The second terminal-end-surface 111 may define the lower end of the accommodation space S. Referring to FIGS. 4 and 5, the second terminal-end-surface 111 may be provided by an upper surface of a protruding wall 11 provided in the base assembly 10. The accommodation groove 31 may be provided in the form of an annular groove extending in the circumferential direction with respect to the bobbin axis A1. The protruding wall 11 may extend in the circumferential direction with respect to the bobbin axis A1 and may be partially accommodated in the accommodation groove 31. The bottom surface of the accommodation groove 31 (i.e., the first terminal-end-surface 311) and the upper surface of the protruding wall 11 (i.e., the second terminal-end-surface 111) face each other in the vertical direction. The height h1 in the vertical direction of the accommodation space S may vary depending on the vertical movement between the bobbin assembly 30 and the base assembly 10.

Referring to FIGS. 3 to 5, the bobbin assembly 30 rotates about the bobbin axis A1 relative to the base assembly 10, and may vertically move relative to the base assembly 10 in a direction parallel to the bobbin axis A1 (or along the bobbin axis A1). As the bobbin assembly 30 vertically moves, the distance between the upper and lower ends of the accommodation space S changes, and the height h1 of the accommodation space S changes. In the present disclosure, the movement of the bobbin assembly 30 along the bobbin axis A1 (or vertical direction) relative to the base assembly 10 is referred to as vertical movement of the bobbin assembly 30. In the present disclosure, unless otherwise stated, the rotation or vertical movement of the bobbin assembly 30 refers to rotation or vertical movement relative to the base assembly 10.

In an embodiment of the present disclosure, the bobbin assembly 30 vertically moves in association with the amount of rotation about the bobbin axis A1. As the procedure tool T advances, the height of the wound portion decreases, and the bobbin assembly 30 descends, so that the height of the accommodation space S decreases by a length corresponding to the reduced height of the wound portion. Conversely, as the procedure tool T retracts and the height of the wound portion increases, the bobbin assembly 30 ascends, so that the height of the accommodation space S increases by a length corresponding to the increased height of the wound portion.

Referring to FIG. 4, as the bobbin assembly 30 rotates once in one direction, the number of windings of the wound portion decreases by one, and the height h2 of the wound portion decreases. As the height h2 of the wound portion decreases, the bobbin assembly 30 descends, and the height hl of the accommodation space S decreases in correspondence with the height h2 of the wound portion. Referring to FIG. 5, as the bobbin assembly 30 rotates once in the direction opposite to that in FIG. 4, the number of windings of the wound portion increases by one, and the height h2 of the wound portion increases. As the height h2 of the wound portion increases, the bobbin assembly 30 ascends, and the height h1 of the accommodation space S increases in correspondence with the height h2 of the wound portion.

If the height h1 of the accommodation space S is fixed, as the height h2 of the wound portion decreases as the procedure tool T is unwound, the empty space in the accommodation space S that is not occupied by the wound portion increases. Insertion of the procedure tool T into a blood vessel (or extraction from the procedure tool module 1) is implemented by the inner end of the procedure tool T, which is fixed to the inside of the bobbin assembly 30, pushing another portion of the procedure tool T. On the other hand, extraction of the procedure tool T from the blood vessel (or insertion into the procedure tool module 1) is implemented when the inner end of the procedure tool T pulls another portion of the procedure tool T. However, when attempting to insert or extract the procedure tool T into or from a blood vessel, if the empty space not occupied by the wound portion in the accommodation space S is excessively great, a delay may occur between the rotation of the bobbin assembly 30 and the movement of the terminal end TE of the procedure tool T during the transition between insertion and extraction of the procedure tool T.

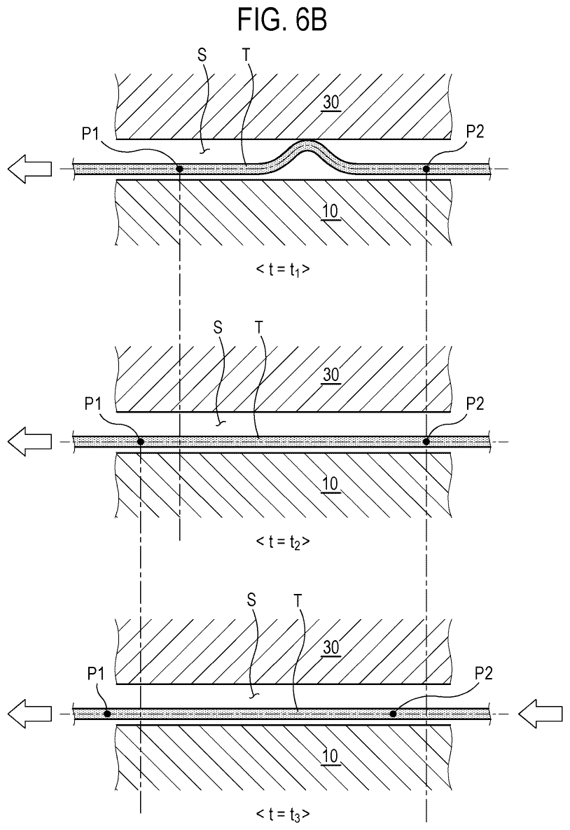

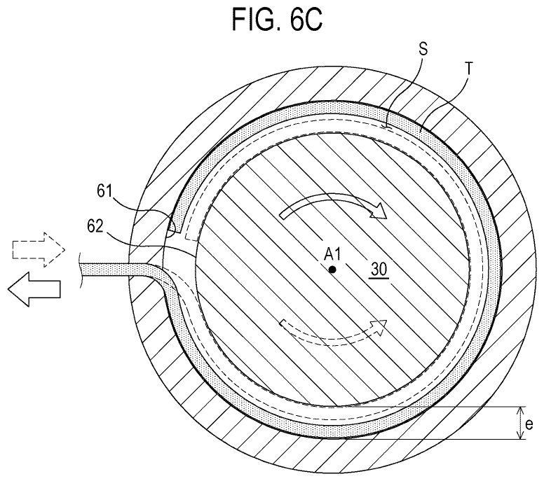

FIG. 6A illustrates the movement of the procedure tool T in the accommodation space S during the advancing process of the procedure tool T in a comparative example. At a first time point t1, a first point P1 (i.e., a point of the procedure tool) of the procedure tool T begins to push another portion of the procedure tool T in the direction of the arrow (i.e., to the right). If the height h1 of the accommodation space S is excessively greater than the thickness of the procedure tool T, then between the first time point t1 and a second time point t2, while the first point P1 of the procedure tool T is pushed to the right, the portion between the first point P1 and a second point P2 (i.e., another point of the procedure tool) bends (i.e., buckles) in the empty portion of the accommodation space S, and the second point P2 of the procedure tool T does not move. The second point P2 of the procedure tool T begins to move to the right only at a third time point t3 when the portion between the first point P1 and the second point P2 no longer bends.

FIG. 6B illustrates the movement of the procedure tool T in the accommodation space S during a retraction process after the procedure tool T has advanced in a comparative example. When the procedure tool T advances, a portion between the first point P1 and the second point P2 of the procedure tool T is already bent and placed in the accommodation space S. At the first time point t1, the first point P1 of the procedure tool T begins to pull another portion in the direction of the arrow (i.e., to the left). If the height h1 of the accommodation space S is excessively greater than the thickness of the procedure tool T, then between the first time point t1 and the second time point t2, while the first point P1 of the procedure tool T is pulled to the left, the bent portion between the first point P1 and the second point P2 straightens, and the second point P2 does not move. The second point P2 is pulled to the left and begins to move only at the third time point t3, when the bent portion of the procedure tool T is completely straightened.

In the present disclosure, in order to solve the above-described problem, the height of the accommodation space S of the procedure tool T is adjusted to correspond to a change in the height of the wound portion of the procedure tool T. Accordingly, during the transition between advancement and retraction of the procedure tool T, the delay between the rotation of the bobbin assembly 30 and the advancement/retraction of the procedure tool T may be prevented or minimized.

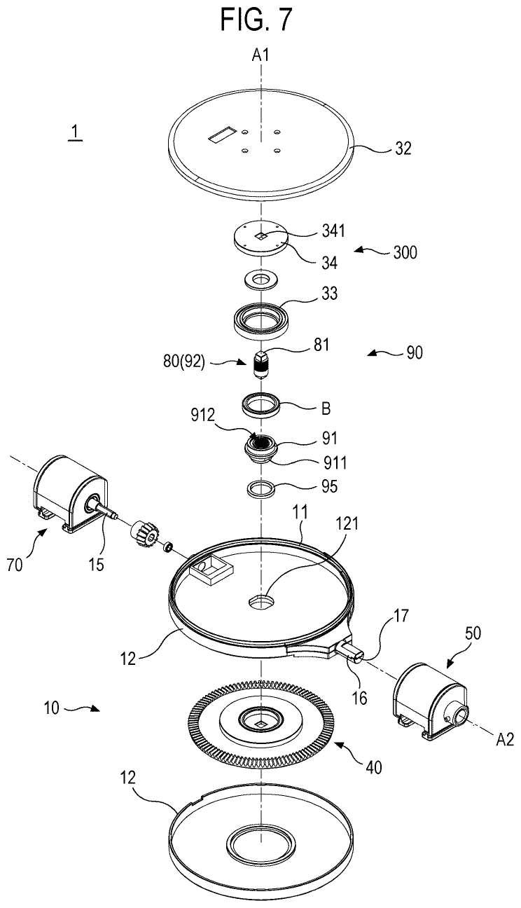

The operation in which the bobbin assembly 30 vertically moves while rotating about the bobbin axis A1 relative to the base assembly 10 may be provided by a vertical moving mechanism (e.g., the vertical moving mechanism 90 of FIG. 7). The vertical moving mechanism is configured to raise and lower the bobbin assembly 30 in the direction of the bobbin axis A1 relative to the base assembly 10, in association with the amount of rotation of the bobbin assembly 30 about the bobbin axis A1.

The vertical moving mechanism may be configured such that the bobbin assembly 30 moves up or down by a fixed height relative to the base assembly 10 whenever the bobbin assembly 30 rotates once relative to the base assembly 10. Since the wound portion of the procedure tool T is wound within the bobbin assembly 30 about the bobbin axis A1, the number of windings of the procedure tool T increases or decreases by one every time the bobbin assembly 30 rotates once. The change in the height of the wound portion per rotation of the bobbin assembly 30 corresponds to the cross-sectional thickness of the procedure tool T. The vertical moving amount of the bobbin assembly 30 by the vertical moving mechanism may be set to correspond to the cross-sectional thickness of the procedure tool T. For example, when the cross-sectional thickness of the procedure tool T is 0.33 mm, the vertical moving mechanism may be configured to vertically move by 0.4 mm per rotation of the bobbin assembly 30. As another example, when the cross-sectional thickness of the procedure tool Tis 0.82 mm, the vertical moving mechanism may be configured to vertically move by 1.0 mm per rotation of the bobbin assembly 30.

Meanwhile, FIG. 6C illustrates radial movement of the procedure tool T within the accommodation space S in a comparative example. In the comparative example of FIG. 6C, the accommodation space S has a radial width e that is excessively greater than the thickness of the procedure tool T. Here, when the bobbin assembly 30 rotates clockwise to insert the procedure tool T into a blood vessel, the procedure tool T moves close to a radially outer surface 61 of the accommodation space S. Conversely, when the bobbin assembly 30 rotates counterclockwise to extract the procedure tool T from the blood vessel, the procedure tool T moves close to a radially inner surface 62 of the accommodation space S. That is, during the transition between insertion (or advancement) and extraction (or retraction) of the procedure tool T, the procedure tool T may move in the radial direction with respect to the bobbin axis A1 within the accommodation space S. As a result, a delay may occur between the rotation of the bobbin assembly 30 and the advancement/retraction of the procedure tool T.

Referring to FIGS. 4 and 5, in the procedure tool module 1 of the present disclosure, in order to prevent or minimize the above-described delay, the radial width d of the accommodation space S may be set to correspond to the cross-sectional thickness of the procedure tool T disposed in the accommodation space S. That is, the width d of the accommodation space S may be determined to minimize radial clearance of the procedure tool T within the accommodation space S to the necessary extent.

The above descriptions of the procedure tool module 1, the base assembly 10, the bobbin assembly 30, the procedure tool T, the vertical moving mechanism, and the interactions among them are also applied to the embodiments described below.

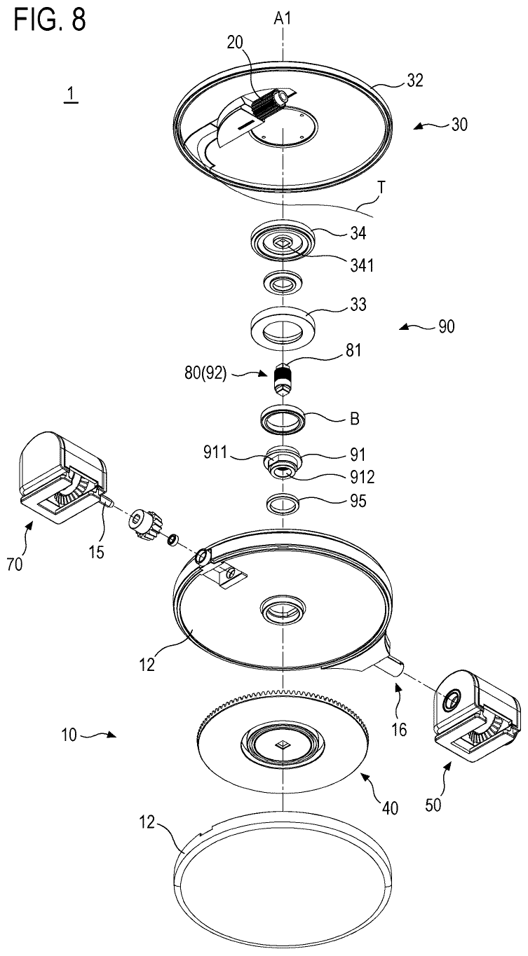

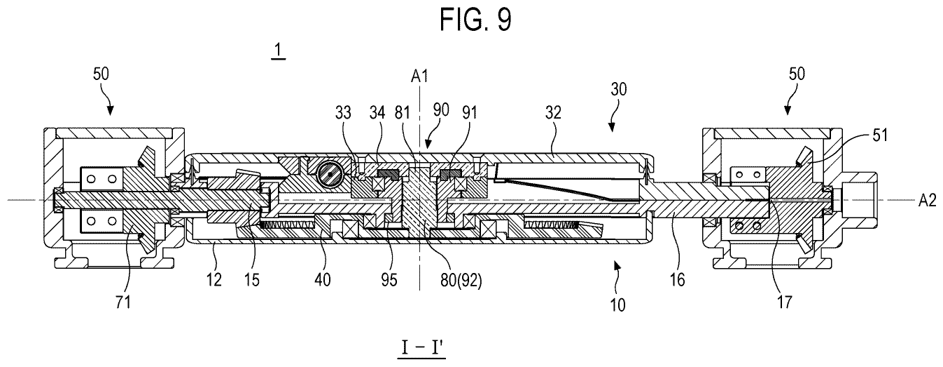

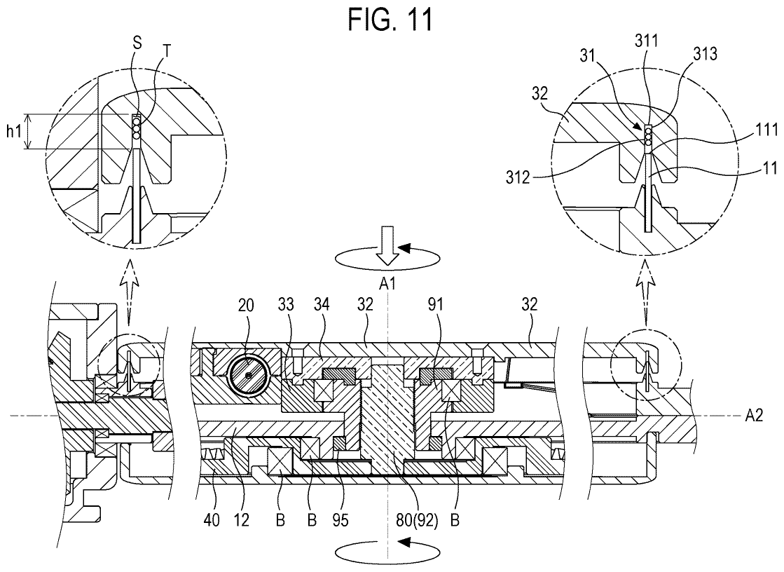

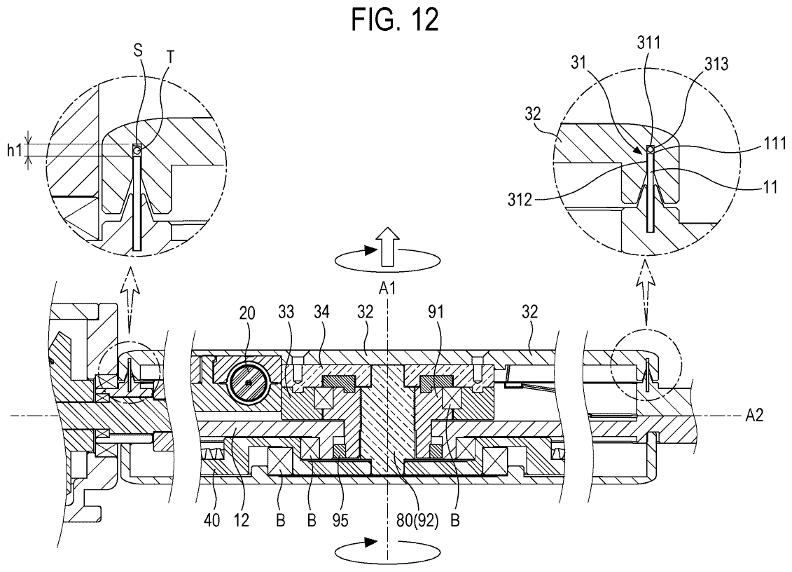

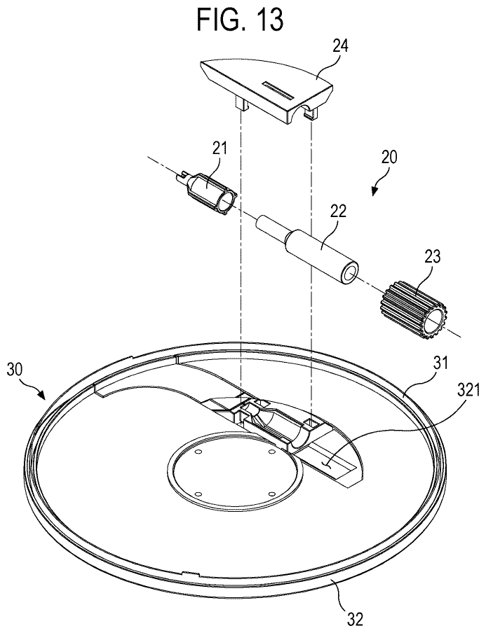

FIG. 7 is an exploded perspective view of the procedure tool module 1 according to an embodiment. FIG. 8 is a rear exploded perspective view of the procedure tool module 1 according to an embodiment. FIG. 9 is a cross-sectional view taken along line I-I′ of FIG. 13. FIG. 10 is an exploded perspective view of the vertical moving mechanism 90 according to an embodiment. FIG. 11 illustrates a rotation and descending operation of the bobbin assembly 30. FIG. 12 illustrates a rotation and ascending operation of the bobbin assembly 30.

Referring to FIGS. 7 to 9, the procedure tool module 1 includes a bobbin assembly 30 rotatably mounted on the base assembly 10 about a bobbin axis Al. The bobbin assembly 30 is mounted on the base assembly 10 to be movable in an vertical moving direction parallel to the bobbin axis A1.

The procedure tool module 1 may include a vertical moving mechanism 90 configured to raise and lower the bobbin assembly 30 in the vertical moving direction relative to the base assembly 10 in association with the amount of rotation of the bobbin assembly 30 about the bobbin axis A1.

The base assembly 10 includes a base body 12 that forms an exterior appearance. The base body 12 may define an internal space configured to accommodate a rotation body 40. The base body 12 may include a plurality of parts assembled together.

The procedure tool module 1 may include the rotation body 40 that is rotatably mounted on the base body 12 of the base assembly 10 about the bobbin axis A1. The rotation body 40 may be mounted on the base body 12 via a bearing B. The rotation body 40 is powered by a bobbin drive shaft 15 and rotates the bobbin assembly 30. The rotation body 40 rotates the bobbin assembly 30 through a rotation pin 80 extending along the bobbin axis A1. The base body 12 includes a base shaft 16. The base shaft 16 is connected to a base driver 50.

The bobbin assembly 30 is coupled to the base assembly 10 to be rotatable about the bobbin axis A1. The bobbin assembly 30 may be coupled to the rotation body 40 so as to have a degree of freedom of motion that allows movement only in the vertical moving direction parallel to the bobbin axis A1 with respect to the rotation body 40. That is, the bobbin assembly 30 rotates together with the rotation body 40 about the bobbin axis A1, but is coupled to the base assembly 10 to be movable in the vertical moving direction parallel to the bobbin axis A1 with respect to the rotation body 40.

In an embodiment, the procedure tool module 1 may include a rotation pin 80 that extends along the bobbin axis A1 from the rotation body 40 and is coupled to the bobbin assembly 30 to be movable in the vertical moving direction. The rotation pin 80 may be provided as a separate member and coupled to the rotation body 40, or may be integrally formed with the rotation body 40. The rotation pin 80 is coupled to the bobbin assembly 30 so as to have one degree of freedom of motion in the direction of the bobbin axis A1. Referring to FIGS. 7 and 10, the rotation pin 80 includes an insertion portion 81, and the bobbin assembly 30 may include a pin accommodation portion 341 into which the insertion portion 81 is fitted. A cross-section of the insertion portion 81 perpendicular to the bobbin axis Al is non-axisymmetric, and the pin accommodation portion 341 may be provided as a hole having a shape corresponding to the insertion portion 81. For example, the insertion portion 81 may have a rectangular column shape, and the pin accommodation portion 341 may be a rectangular hole. The pin accommodation portion 341 may be provided as a separate member (e.g., the second connection member 34 of FIG. 10) coupled to the bobbin body 32, or may be integrally formed with the bobbin body 32.

The vertical moving mechanism 90 may include a vertical moving portion 91 configured to move together with the bobbin assembly 30 in the vertical moving direction, and a vertical moving guide portion 92 configured to move the vertical moving portion 91 in the vertical moving direction relative to the base assembly 10. One of the vertical moving guide portion 92 and the vertical moving portion 91 may include a vertical moving grove extending helically about the bobbin axis A1. The other of the vertical moving guide portion 92 and the vertical moving portion 91 may include a vertical moving protrusion that is at least partially accommodated in the vertical moving grove and configured to be guided along the vertical moving grove when the vertical moving guide portion 92 and the vertical moving portion 91 rotate relative to each other. For example, the vertical moving grove and the vertical moving protrusion may be provided in complementary thread shapes.

Referring to FIGS. 7 to 10, the vertical moving portion 91 is mounted to be movable along the bobbin axis Al relative to the base assembly 10, but its rotation relative to the base assembly 10 is restricted. For example, the vertical moving portion 91 may include an asymmetric slide portion 911, and the slide portion 911 may be fitted into a base hole 121 formed in the base body 12. For example, the cross-section of the slide portion 911 may have a D-cut shape. The base hole 121 may be formed in a shape corresponding to the slide portion 911 so that the slide portion 911 is configured to be movable only along the bobbin axis A1 within the base hole 121. The illustrated shapes of the slide portion 911 and the base hole 121 are merely examples of a coupling structure that allows the vertical moving portion 91 to move only in the vertical moving direction relative to the base assembly 10, and in other embodiments, the slide portion 911 and the base hole 121 may have different shapes. For example, the slide portion 911 may be provided in a polygonal column shape, and the base hole 121 may be provided in a polygonal hole shape.

A stopper 95 is mounted at a lower end of the vertical moving portion 91 to prevent the vertical moving portion 91 from being separated upward from the base body 12. After the vertical moving portion 91 is fitted into the base hole, the stopper 95, having an area greater than the base hole 121, may be mounted at the lower end of the vertical moving portion 91 that protrudes below the base hole 121.

The vertical moving portion 91 is coupled to the bobbin assembly 30 such that vertical movement is restricted, while rotation is allowed. Referring to FIG. 9, an annular first connection member 33 is rotatably coupled to the outer circumferential surface of the vertical moving portion 91, and the first connection member 33 and the bobbin body 32 are fixed to each other via a second connection member 34. A bearing B may be mounted between the first connection member 33 and the vertical moving portion 91. A pin accommodation portion 341, which is slidably coupled to the rotation pin 80 in the direction of the bobbin axis A1, may be defined by the second connection member 34. Although the bobbin body 32, the first connection member 33, and the second connection member 34 are illustrated as separate members, some or all of them may be integrally formed.

Referring to FIGS. 7 to 10, the rotation pin 80 may function as the vertical moving guide portion 92. The vertical moving portion 91 may include a pin hole 912 through which the rotation pin 80 passes. One of the outer circumferential surface of the rotation pin 80 and the inner circumferential surface of the pin hole 912 may include a vertical moving grove, and the other may include a vertical moving protrusion. In the following description, the vertical moving grove is described as being formed on the rotation pin 80, and the vertical moving protrusion is formed on the pin hole for convenience of description, but in other embodiments, the vertical moving grove may be formed on the pin hole, and the vertical moving protrusion may be formed on the rotation pin 80.

Referring to FIG. 10, the outer circumferential surface of the rotation pin 80 (or the vertical moving guide portion 92) may include a male screw 94 as an example of the vertical moving grove, and the inner circumferential surface of the pin hole 912 of the vertical moving portion 91 may include a female screw 93 that meshes with the male screw 94 of the rotation pin 80 as an example of the vertical moving protrusion. The vertical moving portion 91 moves only in the vertical moving direction relative to the base assembly 10, and its rotation relative to the base assembly 10 is restricted. As the vertical moving guide portion 92 rotates, the vertical moving portion 91 may vertically move relative to the base assembly 10 in the up and down direction. For example, the vertical moving portion 91 may descend when the vertical moving guide portion 92 rotates in one direction about the bobbin axis A1, and may ascend when the vertical moving guide portion 92 rotates in the opposite direction.

When the rotation pin 80 is fitted into the pin accommodation portion 341 of the bobbin assembly 30 and then rotates, the bobbin assembly 30 rotates together, and the vertical moving portion 91 connected to the bobbin assembly 30 by the vertical moving mechanism 90 vertically moves. That is, as the vertical moving guide portion 92 (or the rotation pin 80) rotates, the bobbin assembly 30 may vertically move while rotating about the bobbin axis A1.

Referring to FIGS. 11 and 12, the bobbin assembly 30 and the base assembly 10 may define an accommodation space S therebetween in which a procedure tool T is placed. The accommodation space S is configured such that the procedure tool T is wound in the circumferential direction with respect to the bobbin axis Al. The bobbin assembly 30 may include a first terminal-end-surface 311 defining an upper end (or a terminal end in a first direction of the vertical moving direction) of the accommodation space S. The base assembly 10 may include a second terminal-end-surface 111 defining a lower end (or a terminal end in a second direction opposite to the first direction of the vertical moving direction) of the accommodation space S. For example, the bottom surface of the accommodation groove 31 of the bobbin assembly 30 may define the first terminal-end-surface 311, and an upper end of the protruding wall 11 of the base assembly 10 may define the second terminal-end-surface 111.

When the bobbin assembly 30 vertically moves relative to the base assembly 10 by the vertical moving mechanism 90, a height hl of the accommodation space S (i.e., a length of the accommodation space S along the vertical moving direction) may be changed.

The bobbin assembly 30 may include a first wall surface 312 defining a terminal end in the radial inner direction with respect to the bobbin axis Al of the accommodation space S.

The accommodation space S may extend in a circumferential direction with respect to the bobbin axis A1 and may be configured such that the procedure tool T is wound helically on the first wall surface 312.

The bobbin assembly 30 may include a second wall surface 313 defining a terminal end in the radial outer direction with respect to the bobbin axis Al of the accommodation space S. The second wall surface 313 is configured to surround an outer circumference of the wound portion of the procedure tool T disposed in the accommodation space S. In the drawings of the present disclosure, the second wall surface 313 is provided by the bobbin assembly 30. However, this is merely an example, and in other embodiments, the second wall surface 313 may be provided by the base assembly 10. For example, a structure extending from the base assembly 10 may define the terminal end in the radial outer direction of the accommodation space S and may surround the outer circumference of the wound portion.

The base assembly 10 may include a protruding wall 11 extending in the circumferential direction with respect to the bobbin axis A1. The protruding wall 11 may be provided as a separate member and coupled to the base body 12 or may be integrally formed with the base body 12. The protruding wall 11 may be at least partially accommodated between the first wall surface 312 and the second wall surface 313. An end of the protruding wall 11 may provide the second terminal-end-surface 111 defining a lower end of the accommodation space S.

The first terminal-end-surface 311, the first wall surface 312, and the second wall surface 313 may extend in the circumferential direction with respect to the bobbin axis A1, and the protruding wall 11 may also extend in the circumferential direction with respect to the bobbin axis A1. For example, the accommodation groove 31 may have a circular annular groove shape, and the protruding wall 11 may have a circular rib shape.

Referring to FIG. 10, the base may include a guide passage 112 connected to the accommodation space S to guide the procedure tool T. For example, the guide passage 112 may be defined by the protruding wall 11. The guide passage 112 may extend obliquely from the upper end to the lower end of the protruding wall 11. When the bobbin assembly 30 rotates in a direction to unwind the procedure tool T, the procedure tool T wound in the accommodation space S may move along the guide passage 112 from the upper end to the lower end of the protruding wall 11 and then exit to the outside of the procedure tool module 1. When the bobbin assembly 30 rotates in a direction to wind the procedure tool T, a portion of the procedure tool T may move along the guide passage 112 from the lower end to the upper end of the protruding wall 11 and enter the accommodation space S.

Referring to FIGS. 11 and 12, the height h1 of the accommodation space S is defined by the distance between the first terminal-end-surface 311 of the bobbin assembly 30 and the second terminal-end-surface 111 of the base assembly 10. Therefore, as the bobbin assembly 30 vertically moves relative to the base assembly 10, the height h1 of the accommodation space S changes. When the bobbin assembly 30 moves in the upward direction (or the first direction) relative to the base assembly 10, the height h1 of the accommodation space S increases. When the bobbin assembly 30 moves in the downward direction (or the second direction opposite to the first direction) relative to the base assembly 10, the height h2 of the accommodation space S decreases.

The bobbin assembly 30 vertically moves relative to the bobbin assembly 30 while rotating due to the vertical moving mechanism 90. When the bobbin assembly 30 rotates in the first rotation direction about the bobbin axis A1, causing the procedure tool T to be wound onto the bobbin assembly 30, the bobbin assembly 30 moves in the first direction relative to the base assembly 10, and the height hl of the accommodation space S increases. When the bobbin assembly 30 rotates in the opposite direction (a second rotation direction) causing the procedure tool T to be unwound from the bobbin assembly 30, the bobbin assembly 30 moves in the second direction relative to the base assembly 10, and the height h1 of the accommodation space S decreases.

Referring to FIGS. 10 and 11, as the rotation body 40 rotates counterclockwise, the bobbin assembly 30 also rotates counterclockwise together with the rotation body 40. Since the rotation pin 80 moves only in the vertical moving direction relative to the bobbin assembly 30, the bobbin assembly 30 also rotates in accordance with the rotation of the rotation pin 80. When the rotation pin 80 rotates, the rotation pin 80 is inserted into the vertical moving portion 91. Since the male screw 94 is formed on the outer circumferential surface of the rotation pin 80 and the female screw 93 is formed in the pinhole 912 of the vertical moving portion 91, the rotation pin 80 is inserted into the pin hole 912 of the vertical moving portion 91 in a manner similar to a bolt-nut engagement when the rotation pin 80 rotates counterclockwise. That is, as the rotation pin 80 rotates, the vertical moving portion 91 descends, and the bobbin assembly 30 descends together with the vertical moving portion 91. As the rotation pin 80 rotates counterclockwise, the bobbin assembly 30 descends while rotating counterclockwise. As the bobbin assembly 30 rotates counterclockwise, the height of the wound portion of the procedure tool T decreases, and the height h1 of the accommodation space S also decreases accordingly.

Referring to FIGS. 10 and 12, as the rotation body 40 rotates clockwise, the bobbin assembly 30 also rotates clockwise together with the rotation body 40. Since the rotation pin 80 moves only in the vertical moving direction relative to the bobbin assembly 30, the bobbin assembly 30 also rotates in accordance with the rotation of the rotation pin 80. When the rotation pin 80 rotates, the rotation pin 80 is withdrawn from the vertical moving portion 91. In a manner similar to a bolt-nut engagement, when the rotation pin 80 rotates clockwise, the rotation pin 80 is withdrawn from the pin hole 912 of the vertical moving portion 91. That is, as the rotation pin 80 rotates clockwise, the vertical moving portion 91 ascends, and the bobbin assembly 30 ascends together with the vertical moving portion 91. As the rotation pin 80 rotates clockwise, the height of the wound portion of the procedure tool T increases, and the height h1 of the accommodation space S also increases accordingly.

The pitch of the screws 93 and 94 of the vertical moving guide portion 92 and the vertical moving portion 91 may be set in consideration of the thickness of the procedure tool T wound around the bobbin assembly 30. When the vertical moving guide portion 92 rotates once, the vertical moving portion 91 and the bobbin assembly 30 vertically move by a distance corresponding to the pitches of the screws 93 and 94. In addition, when the vertical moving guide portion 92 rotates once, the bobbin assembly 30 also rotates once, so that the height of the wound portion of the procedure tool T increases or decreases by a length corresponding to the thickness of the cross-section of the procedure tool T. By setting the pitches of the screws 93 and 94 to a length corresponding to the cross-section of the procedure tool T, the height of the accommodation space S may appropriately change in response to changes in the height of the wound portion of the procedure tool T as the procedure tool T is unwound or wound.

For example, when the cross-sectional thickness of the procedure tool T is 0.33 mm, the vertical moving mechanism 90 may be configured to vertically move by 0.4 mm per rotation of the bobbin assembly 30. This may be implemented by designing the pitches of the screws 93 and 94 of the vertical moving guide portion 92 and the vertical moving portion 91 to be 0.4 mm. In another example, when the cross-sectional thickness of the procedure tool T is 0.82 mm, the vertical moving mechanism 90 may be configured to vertically move by 1.0 mm per rotation of the bobbin assembly 30. This may be implemented by designing the pitches of the screws 93 and 94 of the vertical moving guide portion 92 and the vertical moving portion 91 to be 1.0 mm.

In the above embodiment, the procedure tool T is described as being unwound when the bobbin assembly 30 rotates counterclockwise and as being wound when the bobbin assembly 30 rotates clockwise. However, this is merely an example, and in other embodiments, the rotation direction of the bobbin assembly 30 and the unwinding/winding direction of the procedure tool T may be configured differently. For example, the procedure tool T may be wound when the bobbin assembly 30 rotates counterclockwise and may be unwound when the bobbin assembly 30 rotates clockwise. In this case, the vertical moving mechanism 90 may be configured to raise the bobbin assembly 30 when the bobbin assembly 30 rotates counterclockwise and to lower the bobbin assembly 30 when the bobbin assembly 30 rotates clockwise.

Referring to FIGS. 7 to 9, the procedure tool module 1 may include a base driver 50 configured to rotate the base assembly 10. The base driver 50 may rotate the base assembly 10 about a base axis A2, which is perpendicular to the bobbin axis A1. The base assembly 10 may include a base shaft 16 extending along the base axis A2, which is perpendicular to the bobbin axis A1, and the base driver 50 may transmit power to the base shaft 16. The base shaft 16 may include an access hole 17 that communicates with the accommodation space S and opens in the direction of the base axis A2. The accommodation space S and the access hole 17 communicate with each other through the guide passage 112. As the bobbin assembly 30 rotates relative to the base assembly 10, the procedure tool T may be extracted through the access hole 17 or inserted into the procedure tool module 1 through the access hole 17.

The procedure tool module 1 may include a bobbin driver 70 configured to be coupled to the base assembly 10 and to rotate the rotation body 40. The bobbin driver 70 may include a bobbin driving shaft 15 installed in the base assembly 10 to be rotatable about the base axis A2. The bobbin driving shaft 15 is installed in the base assembly 10 such that its rotation axis aligns with the base axis A2. The power transmitted to the bobbin driving shaft 15 may rotate the rotation body 40 through gear engagement. Since the rotation axis of the bobbin driving shaft 15 (i.e., the base axis A2) and the rotation axis of the rotation body 40 (i.e., the bobbin axis A1) are perpendicular to each other, the power of the bobbin driving shaft 15 may be transmitted to the rotation body 40 via a bevel gear pair.

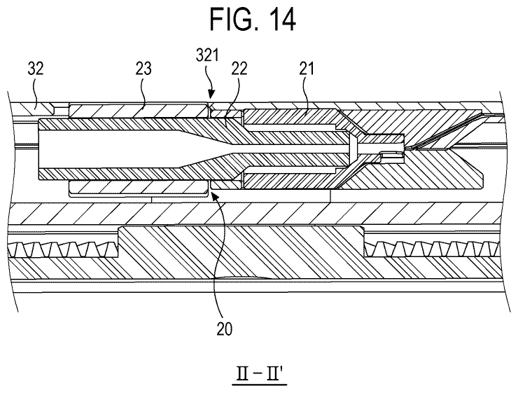

FIG. 13 is an exploded perspective view of a medical torque device 20 according to an embodiment. FIG. 14 is a cross-sectional view taken along line II-II′ of FIG. 2.

Referring to FIGS. 13 and 14, the procedure tool T is mounted to the bobbin assembly 30 through the medical torque device 20. The medical torque device 20 is coupled to one end of the procedure tool T and is mounted to the bobbin assembly 30. The procedure tool T extends from the one end coupled to the medical torque device 20 and is wound into a procedure tool T accommodation groove 31 of the bobbin assembly 30. The bobbin assembly 30 may include a member 24 that fixes the medical torque device 20 to the bobbin body 32.

The medical torque device 20 includes a head portion 21 and a rotation portion 22 inserted into the head portion 21. When the procedure tool T is inserted into the head portion 21 and the rotation portion 22, and the rotation portion 22 is fastened to the head portion 21, the procedure tool T is fixed to the medical torque device 20. For example, the rotation portion 22 may be provided with a male screw, and the head portion 21 may be provided with a female screw, so that the head portion 21 and the rotation portion 22 may be screw-coupled to each other. According to the rotation of the rotation portion 22, the procedure tool T may be fixed to or separated from the medical torque device 20.

The bobbin assembly 30 may include an opening 321 configured to expose at least a portion of the medical torque device 20 to the outside of the bobbin assembly 30. The bobbin body 32 may define the opening 321. An operator may access a portion of the medical torque device 20 from outside the procedure tool module 1 through the opening 321, and may easily release the fastening between the medical torque device 20 and the procedure tool T in an emergency.

A grip portion 23 may be coupled to the rotation portion 22 of the medical torque device 20. At least the grip portion 23 may be exposed to the outside of the procedure tool module 1 through the opening 321. The operator may access the grip portion 23 through the opening 321 and rotate the grip portion 23 to release the coupling between the procedure tool T and the medical torque device 20. The grip portion 23 may include a plurality of protrusions arranged along a circumferential direction and extending in a longitudinal direction (a direction parallel to the rotation axis of the rotation portion 22). The plurality of protrusions may assist the operator in easily rotating the grip portion 23 and the rotation portion 22 through the opening 321.

The procedure tool module 1 and the procedure tool T mounted to the procedure tool module 1 may constitute a procedure tool module set. A portion of the procedure tool T is wound in the accommodation space S inside the procedure tool module 1. As the bobbin assembly 30 rotates about the bobbin axis A1 relative to the base assembly 10, the height of the wound portion of the procedure tool T wound within the bobbin assembly 30 in the vertical moving directiondirection changes. The bobbin assembly 30 vertically moves relative to the base assembly 10 while rotating by the vertical moving mechanism 90, and accordingly, the height of the accommodation space S is also changed to correspond to the height of the wound portion.

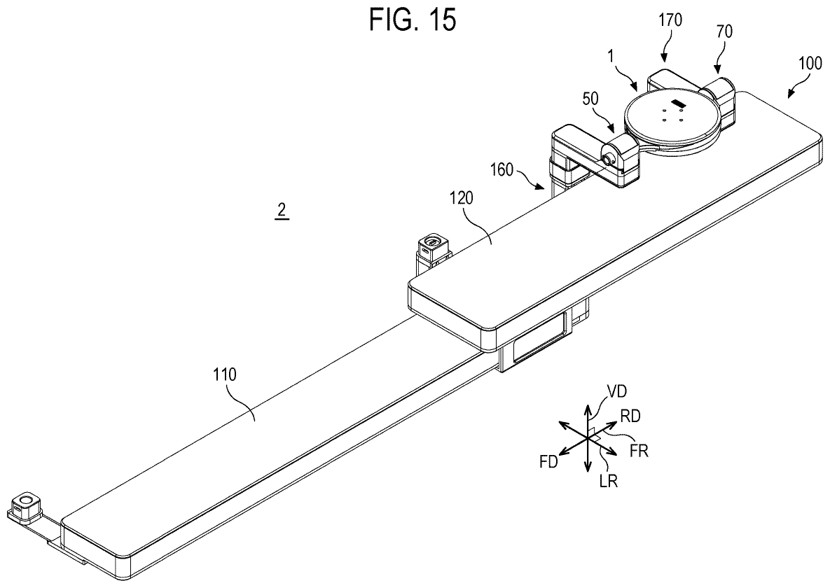

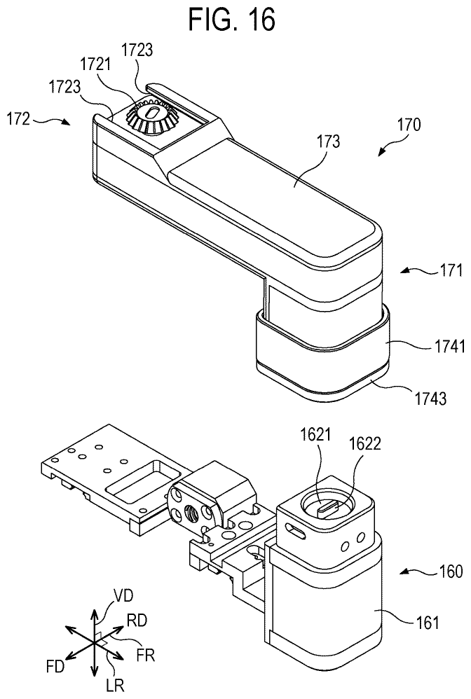

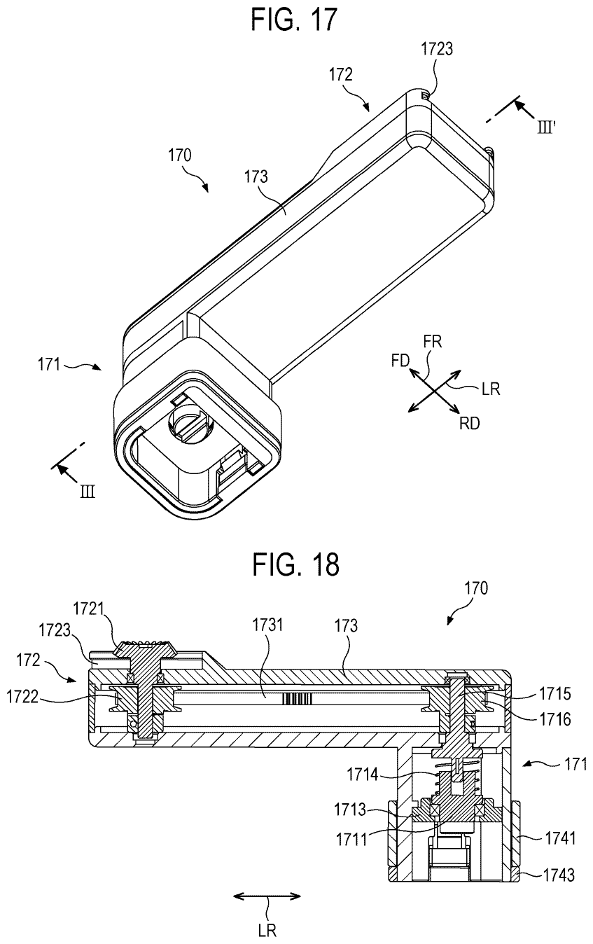

FIG. 15 is a perspective view illustrating a platform 100 of a vascular intervention procedure device 2 according to an embodiment. FIG. 16 is a perspective view illustrating an example of a rotational torque generating portion 160 and a power transmitting portion 170 of FIG. 15. FIG. 17 is a bottom perspective view illustrating the power transmitting portion. FIG. 18 is a cross-sectional view taken along line III-III′ of FIG. 17.

The vascular intervention procedure device 2 may include a procedure tool module 1 and a platform 100 on which the procedure tool module 1 is mounted. The procedure tool module 1 may be detachably coupled to the platform. The platform may include a base platform 110 and a transfer platform 120 slidably coupled to the base platform in a front-rear direction (FR).

The platform 100 may be configured to provide power to the procedure tool module 1. The platform may be configured to transmit a rotational torque to rotate the base assembly 10 of the procedure tool module 1 about the base axis A2. The platform 100 may be configured to transmit a rotational torque to rotate the bobbin assembly 30 of the procedure tool module 1 about the bobbin axis A1 with respect to the base assembly 10. The platform 100 may include a power transmitting portion 170 connected to the base driver 50 and the bobbin driver 70.

Referring to FIGS. 16 and 17, the power transmitting portion 170 includes an input end 171 that receives a rotational torque from the rotational torque generating portion 160 and an output end 172 that outputs the rotational torque to the corresponding procedure tool module 1. The power transmitting portion 170 includes a bridge portion 173 connecting the input end 171 and the output end 172. The input end 171 may be formed such that an output end of the rotational torque generating portion 160 is fitted in a vertical direction. The input end 171 may be positioned on a lateral side of the platform 100. The output end 172 may be positioned above the platform 100. The output end 172 may be detachably coupled to the corresponding procedure tool module 1. Specifically, the drivers 50 and 70 of the procedure tool module 1 may be mounted to the output end 172.

The rotational torque generating portion 160 includes a first rotation body 1621 that outputs a rotational torque at an upper end thereof. The power transmitting portion 170 includes, at the input end 171, a second rotation body 1711 corresponding to the first rotation body 1621. The second rotation body 1711 is configured to have a complementary shape to the first rotation body 1621. One of the first rotation body 1621 and the second rotation body 1711 may have a protrusion, and the other may have a concave portion into which the protrusion is fitted. For example, as illustrated in FIG. 16, the first rotation body 1621 has a protrusion 1622 protruding upward, and as illustrated in FIG. 17, the second rotation body 1711 has a concave portion 1712 into which the protrusion 1622 is fitted.

The power transmitting portion 170 may include a rotation body guide 1713 that holds the second rotation body 1711 at the input end 171 so as to be movable in a vertical direction, so that the power transmitting portion 170 can receive a rotational torque while being detachably coupled to the rotational torque generating portion 160. The power transmitting portion 170 may include a spring 1714 configured to press the second rotation body 1711 toward the first rotation body 1621. The first rotation body 1621 and the second rotation body 1711 may be detachably coupled under the pressing force of the spring 1714. When the input end 171 of the power transmitting portion 170 is coupled to an upper end of the rotational torque generating portion 160, the first rotation body 1621 and the second rotation body 1711 may not be coupled to each other due to their shapes. When the first rotation body 1621 rotates and the first rotation body 1621 and the second rotation body 1711 are aligned to be engageable with each other, the second rotation body 1711 may move toward the first rotation body 1621 along the rotation body guide 1713 by the pressing force of the spring 1714 and may be engaged with the first rotation body 1621.

The power transmitting portion 170 includes a connection shaft 1715 inserted into the second rotation body 1711 and an input timing pulley 1716 coupled to the connection shaft 1715. The spring 1714 may be disposed between the connection shaft 1715 and the second rotation body 1711, and the connection shaft 1715 may be partially inserted into the second rotation body 1711. The second rotation body 1711 and the connection shaft 1715 may be connected using a convex portion and a concave portion. The power transmitting portion 170 includes a drive gear 1721 that outputs a rotation torque. The drive gear 1721 is coupled to an output timing pulley 1722 rotatably disposed at the output end 172. The input timing pulley 1716 and the output timing pulley 1722 are connected by a timing belt 1731 disposed inside the bridge portion 173. The drive gear 1721 may be rotated by the rotational torque of the first rotation body 1621 through belt transmission.

The power transmitting portion 170 includes, at the output end 172, a pair of fitting grooves 1723 configured to be coupled with a corresponding procedure tool module 1. The drive gear 1721 is exposed between a pair of fitting grooves 1723. The pair of fitting grooves 1723 are formed in a lateral direction LR. The procedure tool module 1 has, at an upper end thereof, fitting protrusions configured to be fitted into the fitting grooves 1723 in the lateral direction LR. In another example, the power transmitting portion 170 may have the fitting protrusions, and the procedure tool module 1 may have the fitting grooves.

Each procedure tool module 1 includes a driven gear that is exposed between the pair of fitting protrusions and engaged with the drive gear 1721 of the power transmitting portion. Referring also to FIG. 9, the gear 71 of the bobbin driver 70 and the gear 51 of the base driver 50 may be engaged with the drive gear 1721 disposed at the output end 172.

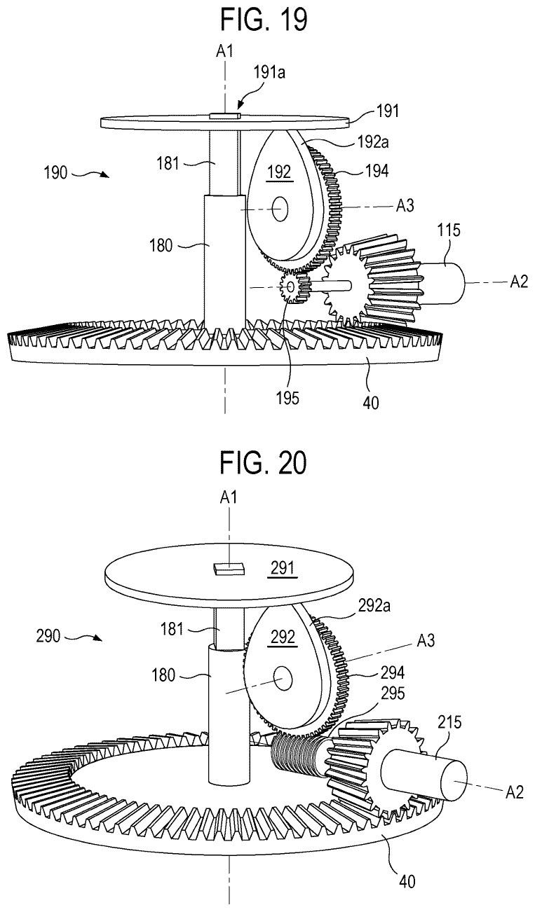

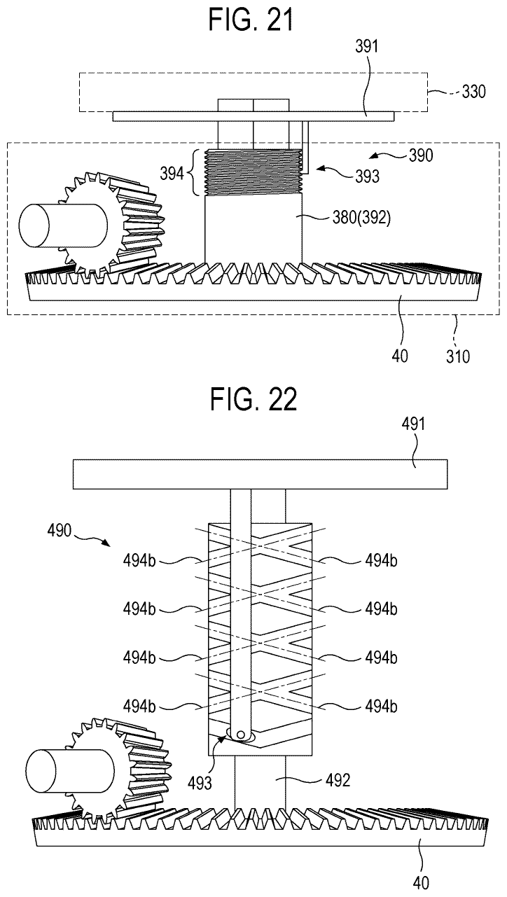

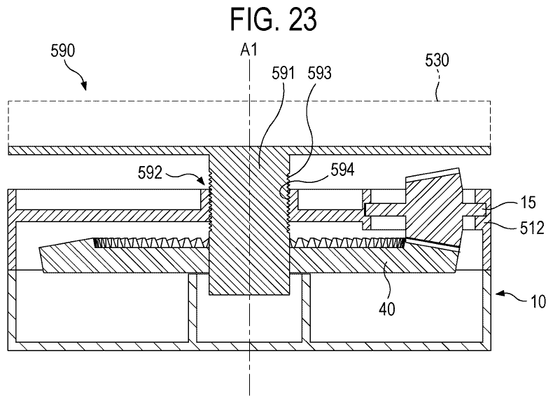

FIG. 19 illustrates a vertical moving mechanism 190 in a first modification. FIG. 20 illustrates a vertical moving mechanism 290 in a second modification. FIG. 21 illustrates a vertical moving mechanism 390 in a third modification. FIG. 22 illustrates a vertical moving mechanism 490 in a fourth modification. FIG. 23 illustrates a vertical moving mechanism 590 in a fifth modification.

Referring to FIG. 19, the vertical moving guide portion 192 of the vertical moving mechanism 190 in the first modification may be a cam that rotates in association with the rotation of a rotation body. That is, the vertical moving guide portion 192 may include a cam surface 192a in which a radial distance from a camshaft A3 changes.

The vertical moving portion 191 may be configured to vertically move by the rotation of the vertical moving guide portion 192. The vertical moving portion 191 may be movable in the vertical direction with respect to the rotation pin 180 and may be coupled to the rotation pin 180 to rotate together with the rotation pin 180. For example, the rotation pin 180 may include a rectangular column 181, and the vertical moving portion 191 may include a rectangular hole 191a corresponding to the rectangular column 181. Although not illustrated, the vertical moving portion 191 is fixedly coupled to the bobbin assembly, and the bobbin assembly may rotate and vertically move together with the vertical moving portion 191.

The vertical moving guide portion 192 is interlocked with the bobbin driving shaft 115 via a pair of spur gears 194 and 195 and rotates around a camshaft A3 parallel to the base shaft A2. As the rotation body 40 rotates, the vertical moving guide portion 192 rotates, and as the vertical moving guide portion 192 rotates, the vertical moving portion 191 rotates and vertically moves. The gear ratio and the shape of the cam surface 192a may be configured such that, when the rotation body 40 makes one rotation, the vertical moving amount of the vertical moving portion 191 by the rotation of the vertical moving guide portion 192 may be configured to match the thickness of the cross-section of the procedure tool T.

Referring to FIG. 20, according to the second modification, the vertical moving mechanism 290 is configured such that a vertical moving guide portion 292 including a cam surface 292a vertically raises and lowers the vertical moving portion 291, as in the first modification. The second modification is distinguished from the first modification in that the direction of the camshaft A3 is not parallel to the bobbin driving shaft 215. For example, the camshaft A3 may be perpendicular to the bobbin driving shaft 215. A worm gear 295 is provided at one end of the bobbin driving shaft 215, and a spur gear 294 attached to the vertical moving guide portion 192 is engaged with the worm gear 295.

Referring to FIG. 21, according to the third modification, the vertical moving guide portion 392 of the vertical moving mechanism 390 may be a rotation pin 380 that extends from the rotation body 40 and includes a male screw 394 as a vertical moving grove on the outer circumferential surface thereof. The vertical moving portion 391 of the vertical moving mechanism 390 may include a vertical moving protrusion 393 received in the male screw 394 provided on the outer circumferential surface of the vertical moving guide portion 392 (or the rotation pin 380). The vertical moving portion 391 may be mounted to the base assembly 310 to be movable in the vertical direction with respect to the base assembly 310 but not rotatable. When the vertical moving guide portion 392 rotates, the vertical moving portion 391 does not rotate, and as the vertical moving protrusion 393 of the vertical moving portion 391 is guided along the male screw 394 of the vertical moving guide portion 392, the vertical moving portion 391 vertically moves. The rotation pin 380 is coupled to the bobbin assembly 330 to be movable only in the vertical direction. When the rotation body 40 rotates, the bobbin assembly 330 rotates by the rotation pin 380, and the bobbin assembly 330 vertically moves by the vertical movement of the vertical moving portion 391.

Referring to FIG. 22, a vertical moving mechanism 490 according to the fourth modification is distinguished from the third modification in that the vertical moving grove has a traverse roll shape. A first helical groove 494a and a second helical groove 494b having lead angles in opposite directions may be formed on the outer circumferential surface of the vertical moving guide portion 492. The first helical groove 494a and the second helical groove 494b are connected to each other at opposite ends. As a result, when the vertical moving guide portion 492 rotates in one direction, a vertical moving protrusion 493 connected to the vertical moving portion 491 may descend (or ascend) along the first helical groove 494a, enter the second helical groove 494b at an end point of the first helical groove 494a, and then ascend (or descend) along the second helical groove 494b.

Referring to FIG. 23, a vertical moving portion 591 of a vertical moving mechanism 590 according to the fifth modification may extend along the bobbin axis Al and may include a male screw 593 on an outer circumferential surface thereof. The vertical moving guide portion 592 of the vertical moving mechanism 590 may be a screw hole formed in the base body 512 and may include a female screw 594 engaged with the male screw 593 of the vertical moving portion 591. The vertical moving portion 591 vertically moves and rotates integrally with a bobbin assembly 530, and is coupled to a rotation body 40 to be movable in the vertical moving direction. For example, the vertical moving portion 591 may include a rectangular column, and the rotation body 40 may include a rectangular hole into which the rectangular column is fitted.

In the foregoing, the technical idea of the present disclosure has been described with reference to some embodiments and examples illustrated in the accompanying drawings.

However, it should be understood that various substitutions, modifications, and changes may be made without departing from the technical idea and scope of the present disclosure, as understood by those ordinarily skilled in the technical field to which the present disclosure pertains. In addition, such substitutions, modifications, and changes should be considered as falling within the scope of the appended claims.

1. A procedure tool module comprising:
a base assembly;
a bobbin assembly mounted to the base assembly to be rotatable about a bobbin axis and to be movable in a vertical moving direction parallel to the bobbin axis, and configured such that a flexible wire-type or tube-type procedure tool insertable into a blood vessel is wound in the bobbin assembly in a circumferential direction with respect to the bobbin axis; and
a vertical moving mechanism configured to raise and lower the bobbin assembly in the vertical moving direction with respect to the base assembly in association with an amount of rotation of the bobbin assembly about the bobbin axis,
wherein the bobbin assembly and the base assembly define an accommodation space therebetween such that the procedure tool is disposed in the accommodation space,
wherein the bobbin assembly comprises a first terminal-end-surface defining a terminal end of the accommodation space in a first direction of the vertical moving direction, and
wherein the base assembly comprises a second terminal-end-surface defining a terminal end of the accommodation space in a second direction opposite to the first direction of the vertical moving direction, and
wherein a length of the accommodation space in the vertical moving direction between the terminal end in the first direction and the terminal end in the second direction changes according to vertical movement of the bobbin assembly in the vertical moving direction with respect to the base assembly.

2. The procedure tool module of claim 1, wherein the length of the accommodation space in the vertical moving direction increases when the bobbin assembly moves in the first direction with respect to the base assembly, and decreases when the bobbin assembly moves in the second direction with respect to the base assembly.

3. The procedure tool module of claim 2, wherein the bobbin assembly is configured to:
move in the first direction with respect to the base assembly when the bobbin assembly rotates in a first rotation direction about the bobbin axis to wind the procedure tool within the bobbin assembly; and
move in the second direction with respect to the base assembly when the bobbin assembly rotates in a second rotation direction opposite to the first rotation direction to unwind the procedure tool from the bobbin assembly.

4. The procedure tool module of claim 1, wherein the accommodation space extends in a circumferential direction with respect to the bobbin axis of the bobbin assembly.

5. The procedure tool module of claim 1, wherein the bobbin assembly comprises a first wall surface defining a terminal end of the accommodation space in a radially inner direction with respect to the bobbin axis.

6. The procedure tool module of claim 5, wherein the accommodation space extends in a circumferential direction with respect to the bobbin axis, and the procedure tool is configured to be wound helically on the first wall surface.

7. The procedure tool module of claim 5, wherein the bobbin assembly comprises a second wall surface defining a terminal end of the accommodation space in a radially outer direction with respect to the bobbin axis.

8. The procedure tool module of claim 7, wherein the base assembly comprises a protruding wall configured to be at least partially accommodated between the first wall surface and the second wall surface and to define the second terminal-end-surface.

9. The procedure tool module of claim 8, wherein the first terminal-end-surface, the first wall surface, and the second wall surface extend in a circumferential direction with respect to the bobbin axis, and
wherein the protruding wall extends in the circumferential direction with respect to the bobbin axis.

10. The procedure tool module of claim 1, wherein the base assembly comprises a guide passage connected to the accommodation space to guide the procedure tool.

11. The procedure tool module of claim 1, further comprising a rotation body rotatably mounted to the base assembly about the bobbin axis,
wherein the bobbin assembly is rotatable about the bobbin axis together with the rotation body and is mounted to the rotation body to be movable in the vertical moving direction parallel to the bobbin axis with respect to the rotation body.

12. The procedure tool module of claim 1, wherein the vertical moving mechanism comprises:
a vertical moving portion configured to move together with the bobbin assembly in the vertical moving direction, and
a vertical moving guide portion configured to move the vertical moving portion in the vertical moving direction with respect to the base assembly.

13. The procedure tool module of claim 12, wherein one of the vertical moving guide portion and the vertical moving portion comprises a vertical moving groove extending helically about the bobbin axis, wherein the other one of the vertical moving guide portion and the vertical moving portion comprises a vertical moving protrusion configured to be at least partially accommodated in the vertical moving groove and to be guided along the vertical moving groove when the vertical moving guide portion and the vertical moving portion rotate relative to each other.

14. The procedure tool module of claim 11, wherein the vertical moving mechanism comprises:
a vertical moving guide portion comprising a rotation pin extending along the bobbin axis from the rotation body and coupled to the bobbin assembly to be movable in the vertical moving direction; and
a vertical moving portion rotatably coupled to the bobbin assembly about the bobbin axis and comprising a pin hole through which the rotation pin passes,
wherein one of an outer circumferential surface of the rotation pin and an inner circumferential surface of the pin hole comprises a vertical moving groove extending helically about the bobbin axis, and the other one of the outer circumferential surface of the rotation pin and the inner circumferential surface of the pin hole comprises a vertical moving protrusion configured to be at least partially accommodated in the vertical moving groove and to be guided along the vertical moving groove when the vertical moving guide portion and the vertical moving portion rotate relative to each other, and
wherein, as the rotation pin rotates and the vertical moving protrusion is guided along the vertical moving groove, the vertical moving portion is configured to move in the vertical moving direction with respect to the base assembly, and the bobbin assembly is configured to move in the vertical moving direction together with the vertical moving portion while rotating about the bobbin axis.

15. The procedure tool module of claim 14, wherein the bobbin assembly comprises a pin accommodation portion configured to allow movement of the rotation pin in the vertical moving direction with respect to the bobbin assembly while restricting rotation of the rotation pin about the bobbin axis with respect to the bobbin assembly.

16. The procedure tool module of claim 1, wherein the bobbin assembly comprises:
a medical torque device accommodation portion configured to accommodate a medical torque device that clamps one end of the procedure tool; and
an opening configured to expose at least a portion of the medical torque device accommodation portion to an outside of the bobbin assembly.

17. The procedure tool module of claim 1, further comprising a bobbin driver coupled to the base assembly and configured to rotate the bobbin assembly,
wherein the base assembly comprises a base shaft extending along a base axis perpendicular to the bobbin axis, and
wherein the base shaft comprises an access hole that communicates with the accommodation space and opens in a direction of the base axis.

18. A procedure tool module set comprising:
the procedure tool module of claim 1; and
a flexible wire-type or tube-type procedure tool wound within the bobbin assembly and insertable into a blood vessel,
wherein a height of a wound portion of the procedure tool, which is wound within the bobbin assembly, in the vertical moving direction is configured to change as the bobbin assembly rotates about the bobbin axis with respect to the base assembly, and
wherein the vertical moving mechanism is configured to change a height of the accommodation space in the vertical moving direction in correspondence to the height of the wound portion in the vertical moving direction.

19. A vascular intervention procedure device comprising:
the procedure tool module of claim 1; and
a platform to which the procedure tool module is detachably coupled, the platform being configured to transmit a rotational torque to rotate the bobbin assembly to the procedure tool module.

20. The vascular intervention procedure device of claim 19, wherein the platform is configured to rotate the base assembly about a base axis perpendicular to the bobbin axis.
